(19) United States
(12) Patent Application Publication
(10) Pub. No.: US 2018/0110888 A1
(43) Pub. Date: Apr. 26, 2018

(54) COMBINATIONS FOR THE TREATMENT OF CANCER

(71) Applicant: BAYER PHARMA AKTIENGESELLSCHAFT, Berlin (DE)

(72) Inventor: Arne SCHOLZ, Berlin (DE)

(21) Appl. No.: 15/817,041

(22) Filed: Nov. 17, 2017

Related U.S. Application Data
(63) Continuation of application No. 14/654,053, filed on Jun. 19, 2015, now abandoned.
(60) Provisional application No. 61/739,315, filed on Dec. 19, 2012.

(51) Int. Cl. A61K 51/02; A61K 31/58; A61K 51/00; A61K 45/06; A61K 33/24

(52) U.S. Cl. CPC A61K 51/02; A61K 31/58; A61K 51/00; A61K 2300/00; A61K 33/24; A61K 45/06

(57) ABSTRACT

The present invention relates to combinations comprising compounds A and B, compound A being a 17α-hydroxylase/C_{17,20}-lyase (CYP17) inhibitor, and compound B being a pharmaceutically acceptable salt of the alkaline-earth radionuclide radium-223 and their use for the treatment or prophylaxis of a disease, particurlarly for the treatment of cancer.

This application is a continuation of U.S. patent application Ser. No. 14/654,053, filed Jun. 19, 2015, which is a U.S. national phase application of International Patent Application No. PCT/EP2013/076865, filed Dec. 17, 2013, which claims priority to U.S. Provisional Patent Application No. 61/739,315, filed Dec. 19, 2012, the contents of all of which are incorporated herein by reference in their entirety.

The present invention relates to combinations comprising compounds A and B, compound A being a 17α-hydroxylase/C_{17,20}-lyase (CYP17) inhibitor, and compound B being a pharmaceutically acceptable salt of the alkaline-earth radionuclide radium-223.

Further, the present invention relates to a kit comprising a combination of:
component A: one or more compounds A, as defined supra, or a physiologically acceptable salt, solvate, hydrate or stereoisomer thereof;
component B: compound B as defined supra, or a solvate or hydrate thereof; and
component C: one or more pharmaceutical agents;
   in which optionally either or both of said components A and B are in the form of a pharmaceutical formulation which is ready for use to be administered to a patient.

Another aspect of the present invention relates to the use of such combinations as described supra for the treatment or prophylaxis of a disease, particurlarly for the treatment of cancer.

### BACKGROUND OF THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

Prostate cancer (PCA) is currently the most common non-skin cancer and the second leading cause of cancer-related death in men after lung cancer. The primary course of treatment for patients diagnosed with organ-confined prostate cancer is usually prostatectomy or radiotherapy. Not only are these treatments highly invasive and have undesirable side effects, such localized treatments are not effective on prostate cancer after it has metastasized. Moreover, a large percent of individuals who receive localized treatments will suffer from recurring cancer.

Additionally, breast cancer is the most common cancer among white and African-American women. Similar to treating prostate cancer, most options for women diagnosed with breast cancer are highly invasive and have significant side-effects. Such treatments include surgery, radiation and chemotherapy.

Hormone therapy is another treatment option for individuals diagnosed with hormone-dependent, hormone-responsive, or hormone-sensitive cancers, such as prostate or breast cancer. Hormone therapy is a form of systemic treatment for cancers such as prostate or breast cancer wherein hormone ablation agents are used to suppress the production or block the effects of hormones, such as estrogen and progesterone in the body, which are believed to promote the growth of breast cancer, as well as, testosterone and dihydrotestosterone, which are believed to promote the growth of prostate cancer.

Moreover, hormone therapy is less invasive than surgery and does not have many of the side effects associated with chemotherapy or radiation. Hormone therapy can also be used by itself or in addition to localized therapy and has shown to be effective in individuals whose cancer has metastasized.

Androgens play an important role in the development, growth, and progression of PCA (McConnell, J. D., Urol. Clin. North Am., 1991, 18: 1-13), with the two most important androgens in this regard being testosterone, 90% of which is synthesized in the testes and the remainder (10%) is synthesized by the adrenal glands, and the more potent androgen, dihydrotestosterone (DHT), to which testosterone is converted by the enzyme steroid, 5α-reductase, that is localized primarily in the prostate (Bruchovsky, N. et al., J. Biol. Chem., 1968, 243, 2012-2021).

Huggins et al. introduced androgen deprivation as a therapy for advanced and metastatic PCA in 1941 (Huggins, C. et al., Arch. Surg., 1941, 43, 209-212), and since then, androgen ablation therapy has been shown to produce the most beneficial responses in multiple settings in PCA patients (Denmeade, S. R. et al., Nature Rev. Cancer, 2002, 2: 389-396). Orchiectomy (either surgical, or medical with a GnRH agonist) remains the standard treatment option for most prostate cancer patients, reducing or eliminating androgen production by the testes, but not affecting androgen synthesis in the adrenal glands.

Several studies have reported that a combination therapy of orchiectomy with antiandrogens to inhibit the action of adrenal androgens significantly prolongs the survival of PCA patients (Crawford, E. D. et al., New Engl. J. Med., 1989, 321, 419-424; Crawford, E. D. et al., J. Urol., 1992, 147: 417A; and Denis, L., Prostate, 1994, 5 (Suppl.), 17s-22s).

In a recent featured article by Mohler and colleagues (Mohler, J. L. et al., Clin. Cancer Res., 2004, 10, 440-448) it was demonstrated that testosterone and dihydrotestosterone occur in recurrent PCA tissues at levels sufficient to activate androgen receptors. In addition, using microarray-based profiling of isogenic PCA xenograft models, Sawyer and colleagues (Chen, C. D. et al., Nat. Med., 2004, 10, 33-39) found that a modest increase in androgen receptor mRNA was the only change consistently associated with the development of resistance to antiandrogen therapy. Potent and specific compounds that inhibit androgen synthesis in the testes, adrenals, and other tissue may therefore be a more effective for the treatment of PCA (Njar, V.C.O. and Brodie, A. M. H., Current Pharm. Design, 1999, 5: 163-180).

In the testes and adrenal glands, the last step in the biosynthesis of testosterone involves two key reactions that occur sequentially, both reactions being catalyzed by a single enzyme, the cytochrome P450 monooxygenase 17α-hydroxylase/C_{17,20}-lyase (CYP17) (Hall, P. F., J. Steroid Biochem. Molec. Biol., 1991, 40, 527-532). Ketoconazole, an antifungal agent that also inhibits P450 enzymes, is also a modest CYP17 inhibitor, and has been used clinically for the treatment of PCA (Trachtenberg, J. et al., J. Urol. 1983, 130, 152-153). It has been reported that careful scheduling of treatment can produce prolonged responses in otherwise castrate-resistant prostate cancer patients (Muscato, J. J. et al., Proc. Am. Assoc. Cancer Res., 1994, 13: 22 (Abstract)). Further, ketoconazole was found to retain activity in advanced PCA patients with progression, despite flutamide withdrawal (Small, E. J. et al., J. Urol., 1997, 157, 1204-1207), and although the drug has now been withdrawn from use because of liver toxicity and other side effects, the ketoconazole results suggest that more potent and selective inhibitors of CYP17 could provide useful agents for treating this disease, even in advanced stages, and in some patients who may appear to be hormone refractory.

A variety of potent steroidal and non-steroidal inhibitors of CYP17 have been reported, some of which having been shown to be potent inhibitors of testosterone production in rodent models (Njar and Brodie, op. cit.). Jarman and colleagues described the hormonal impact of their most potent CYP17 inhibitor, abiraterone, in patients with prostate cancer (O'Donnell, A. et al., Br. J. Cancer, 2004, 90: 2317-2325). Some potent CYP17 inhibitors have been shown to also inhibit 5α-reductase and/or be potent antiandrogens with potent antitumor activity in animal models (Njar and Brodie, op. cit., and Long, B. J. et al., Cancer Res., 2000, 60, 6630-6640).

Abiraterone inhibits CYP17 with an IC50 of 72 nM, in human testicular microsomes (Hu Q., et al. J. Med. Chem. 2010, 53(15), 5749-5758). CYP17 (17α-hydroxylase/C_{17,20}-lyase) is an enzyme which is expressed in testicular, adrenal, and prostatic tumor tissues. It catalyzes two sequential reactions: (a) the conversion of pregnenolone and progesterone to their 17α-hydroxy derivatives by its 17α-hydroxylase activity, and (b) the subsequent formation of dehydroepiandrosterone (DHEA) and androstenedione, respectively, by its C_{17,20} lyase activity.

In the present commercial preparation abiraterone is formulated as the prodrug abiraterone acetate. After oral administration abiraterone acetate is converted into the active form, abiraterone; this conversion is likely to be esterase-mediated and not CYP-mediated. Administration with food increases absorption of the drug and thus has the potential to result in increased and highly variable exposures; the drug should be consumed in empty stomach. The drug is highly protein bound (>99%), and is metabolised in the liver by CYP3A4 and SULT2A1 to inactive metabolites. The drug is excreted by feces (˜88%) and urine (˜5%) with a terminal half life of 12±5 hours (Zytiga prescribing information, Janssen Biotech. May 2012. zytigahcp.com/pdf/full_prescribing_info.pdf).

Mahajan et al. reported that Androgen deprivation therapy has been the standard of care in prostate cancer due to its effectiveness in initial stages. However, the disease recurs, and this recurrent cancer is referred to as castration-resistant prostate cancer (CRPC). Radiotherapy is the treatment of choice; however, in addition to androgen independence, CRPC is often resistant to radiotherapy, making radioresistant CRPC an incurable disease. The molecular mechanisms by which CRPC cells acquire radioresistance are unclear (J. Biol. Chem. 2012; 287(26): 22112-22).

The problem of radioresistance and molecular mechanisms by which prostate carcinoma cells overcome cytotoxic effects of radiation therapy remains to be elucidated. According to Skvortsova et al. radioresistance development is accompanied by multiple mechanisms, including activation of cell receptors and related downstream signal transduction pathways. Identified proteins regulated in the radioresistant prostate carcinoma cells can significantly intensify activation of intracellular signaling that govern cell survival, growth, proliferation, invasion, motility, and DNA repair (Proteomics 2008; 8(21): 4521-33).

According to Mahajan et al. the radioresistance in CRPC might be reversed by a synergistic approach that includes radiotherapy along with the suppression of Ack1/AR/ATM signaling by the Ack1 inhibitor, AIM-100.

A substantial percentage of cancer patients is affected by skeletal metastases. As many as 85% of patients with advanced lung, prostate and breast carcinoma develop bony metastases (Garret R. Semin. Oncol. 72, 3433 -3435 (1993) Bone destruction in cancer.; Nielsen, O S, Munro A J, Tannock I F. J C lin Oneal 9, 509-5 24 (1991), Bone metastases: Pathophysiology and management policy.). Established treatments such as hormone therapy, chemotherapy and external radiotherapy often causes temporary responses, but ultimately most bone cancer patients experience relapses (Kanis J A. Bone 17,101s-105s (1995), Bone and cancer. Pathophysiology and treatment of metastases.). There is thus a strong need for new therapies to relieve pain and slow down tumor progression.

²²³Ra is used as an α-emitting radiopharmaceutical for targeting of calcified tissues, e.g., bone surfaces and osseous tumor lesions. It can be suitable as a bone seeking radiopharmaceutical.

It thus may be used for prophylactic cancer treatment by delivering a focused dose to bone surfaces in patients with a high probability of having undetected micrometastases at bone surfaces. Another example of its potential use would be in the treatment of painful osseous sites.

The alkaline-earth radionuclide radium-223 is useful for the targeting of calcified tissues, e.g., bone and a physiological acceptable solution comprising ²²³Ra.

The alkaline-earth radionuclide radium-223 is suitable for the use of the nuclide as a cationic species and/or associated to a chelator or another form of a carrier molecule with affinity for calcified tissues. Thus may be combined with a chelator that can be subsequently conjugated to a molecule with affinity for calcified tissues. The effect of the radioisotope to generated by providing a cascade of α-particles on bone surfaces and/or in calcified tumors for the palliation of pain caused by various diseases and/or for the prophylactic use against possible minimal disease to the skeleton, and/or also for the therapeutic treatment of established cancer to the bone. The diseases where the radioisotopes could be used includes, but are not limited to skeletal metastases of prostate-, breast-, kidney- and lung cancer as well as primary bone cancer and also multiple myeloma.

Radium-223 dichloride is a novel, targeted alpha-emitter that selectively binds to areas of increased bone turnover in bone metastases and emits high-energy alpha-particles of extremely short (<100 μm) range (Bruland O. S., Nilsson S., Fisher D. R., et al., High-linear energy transfer irradiation targeted to skeletal metastases by the alpha-emitter ²²³Ra: adjuvant or alternative to conventional modalities?, Clin. Cancer Res. 2006; 12: 6250s-7s).

It is the first targeted alpha-emitter to be evaluated in a phase 3 study.

As a bone-seeking calcium mimetic, radium-223 is bound into newly formed bone stroma, especially within the microenvironment of osteoblastic or sclerotic metastases (Henriksen G., Breistol K., Bruland O. S., et al., Significant antitumor effect from bone-seeking, alpha-particle-emitting (223)Ra demonstrated in an experimental skeletal metastases model, Cancer Res. 2002; 62: 3120-3125; Henriksen G., Fisher D. R., Roeske J. C., et al., Targeting of osseous sites with alpha-emitting 223Ra: comparison with the beta-emitter 89Sr in mice, J. Nucl. Med 2003; 44: 252-59).

The high-energy alpha-particle radiation induces mainly double-strand DNA breaks resulting in a potent and highly localized cytotoxic effect in the target areas containing metastatic cancer cells (Lewington V. J., Bone-seeking radionuclides for therapy, J. Nucl. Med 2005; 46 (suppl 1): 38S-47S; Liepe K., Alpharadin, a 223Ra-based alpha-particle-emitting pharmaceutical for the treatment of bone metastases in patients with cancer, Curr. Opin. Investig. Drugs 2009; 10: 1346-58; McDevitt M. R., Sgouros G., Finn R. D., et al., Radioimmunotherapy with alpha-emitting nuclides, Eur. J. Nucl. Med. 1998; 25: 1341-51.).

The short path length of the alpha-particles also means that toxicity to adjacent healthy tissue and particularly the bone marrow may be reduced (Kerr C., (223)Ra targets skeletal metastases and spares normal tissue, Lancet Oncol. 2002; 3: 453; Li Y., Russell P. J., Allen B. J., Targeted alpha-therapy for control of micrometastatic prostate cancer, Expert Rev. Anticancer Ther. 2004; 4: 459-68).

Radium-223 has demonstrated a favorable safety profile, with minimal myelotoxicity, in phase 1 and 2 studies of patients with bone metastases (Nilsson S., Larsen R. H., Fossa S. D., et al., First clinical experience with alpha-emitting radium-223 in the treatment of skeletal metastases, Clin. Cancer Res. 2005; 11: 4451-59; Nilsson S., Franzen L., Parker C., et al., Bone-targeted radium-223 in symptomatic, hormone-refractory prostate cancer: a randomised, multicentre, placebo-controlled phase II study, Lancet Oncol. 2007; 8: 587-94).

Phase 2 studies have shown that radium-223 reduces pain, improves disease-related biomarkers (e.g., bone alkaline phosphatase [ALP] and prostate-specific antigen [PSA]), and have suggested a survival benefit in patients with CRPC and bone metastases (Parker C., Pascoe S., Chodacki A., et al., A randomized, double-blind, dose-finding, multicenter, phase 2 study of radium chloride (Ra-223) in patients with bone metastases and castration-resistant prostate cancer, Eur. Urol. 2012; September 13. pii: S0302-2838(12)01031-7. doi: 10.1016/j.eururo.2012.09.008. [Epub ahead of print]; Nilsson S., Strang P., Aksnes A. K., et al., A randomized, dose-response, multicenter phase II study of radium-223 chloride for the palliation of painful bone metastases in patients with castration-resistant prostate cancer, Eur. J. Cancer 2012; 48: 678-86.

The ALSYMPCA (ALpharadin in SYMptomatic Prostate CAncer patients) trial provides proof of principle for the role of targeted alpha-emitters in oncology. In this trial, radium-223 significantly prolonged overall survival with a 30.5% reduction in risk of death compared with placebo in patients with CRPC (Castration Resistant Prostate Cancer) and bone metastases. Median survival with radium-223 was longer than placebo by 2.8 months. All main secondary efficacy endpoints were statistically significant and favored treatment with radium-223, including the clinically defined endpoint of time to first skeletal-related event, which was significantly prolonged in patients receiving radium-223.

Despite the progress made in the treatment of cancer there remains a need for more effective ways to treat cancer such as, but not limited to, prostate cancer and breast cancer. Additionally, there is a need for effective anti-cancer treatment options for patients who are not responding to current anti-cancer treatments, such as hormone therapy or chemotherapy. Also there is a need for effective anti-cancer treatment options for patients whose cancer has recurred.

### SUMMARY OF THE INVENTION

The present invention relates to combinations comprising compounds A and B, compound A being a 17α-hydroxylase/C_{17,20}-lyase (CYP17) inhibitor, and compound B being a pharmaceutically acceptable salt of the alkaline-earth radionuclide radium-223.

The combinations comprising compounds A and B, as decribed and defined herein, are also referred to as “combinations of the present invention”; a compound A, as decribed and defined herein, is also referred to as “compound A of the the present invention” and a compound B, as decribed and defined herein, is also referred to as “compound B of the the present invention”, respectively. Compounds A and B jointly are also referred to as “compounds of the present invention”.

Further, the present invention relates to:

a kit comprising:
a combination of:

component A: one or more 17α-hydroxylase/C_{17,20}-lyase (CYP17) inhibitors, or a physiologically acceptable salt, solvate, hydrate or stereoisomer thereof;

component B: a suitable pharmaceutically acceptable salt of the alkaline-earth radionuclide radium-223 or a solvate or a hydrate thereof; and, optionally,

component C: one or more further pharmaceutical agents;

in which optionally either or both of said components A and B in any of the above-mentioned combinations are in the form of a pharmaceutical formulation which is ready for use to be administered to a patient.

The components may be administered independently of one another by the oral, intravenous, topical, local installations, intraperitoneal or nasal route.

In accordance with another aspect, the present invention covers the combinations as described supra for the treatment or prophylaxis of a disease.

In accordance with another aspect, the present invention covers the use of such combinations as described supra for the preparation of a medicament for the treatment or prophylaxis of a disease.

### DETAILED DESCRIPTION OF THE INVENTION

Definitions of Terms Used Herein

The term “17α-hydroxylase/C_{17,20}-lyase inhibitor” as used herein refers to an inhibitor of 17α-hydroxylase/C_{17,20}-lyase inhibitor which is an enzyme in testosterone synthesis, an analog thereof, derivative thereof, metabolite thereof or pharmaceutically acceptable salt thereof. Also, unless otherwise noted, reference to a particular 17α-hydroxylase/C_{17,20}-lyase inhibitor can include analogs, derivatives, metabolites or pharmaceutically acceptable salts of such particular 17α-hydroxylase/C_{17,20}-lyase inhibitor.

The term “CYP 17 inhibitor” is used synonymously to the term “17α-hydroxylase/C_{17,20}-lyase inhibitor” as defined supra.

As used herein, and unless otherwise defined, the phrase “therapeutically effective amount” when used in connection with a 17α-hydroxylase/C_{17,20}-lyase inhibitor means an amount of the 17α-hydroxylase/C_{17,20}-lyase inhibitor effective for treating a disease or disorder disclosed herein, such as cancer.

The phrase “pharmaceutically acceptable salt” when used in connection with a 17α-hydroxylase/C_{17,20}-lyase inhibitor refers to any salt of a 17α-hydroxylase/C_{17,20}-lyase inhibitor which may retain or improve the biological effectiveness of the 17α-hydroxylase/C_{17,20}-lyase inhibitor. Examples of pharmaceutically acceptable salts include, but are not limited to, acetates, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, din itrobenzoates, hydroxybenzoates, methoxybenzoates, phthalates, sulfonates, xylenesulfonates, phylacetates, phenylpropionates, phenylbutyrates, citrates, lactates, gammahydroxybutyrates, glycollates, tartarates, alkanesulfonates (e.g. methane-sulfonate or mesylate), propanesulfonates, naphthalene-1-sulfonates, naphthalene-2-sulfonates, and mandelates. Several of the officially approved salts are listed in Remington: The Science and Practice of Pharmacy, Mack Publ. Co., Easton.

The term “pharmaceutically acceptable” is used synonymously to the term “physiologically acceptable”.

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

The term “about” when used herein in connection with a value X means any value in the range of X−(10% of X) to X+(10% of X), or in other words in the range of 90% of X to 110% of X.

Where the plural form of the word compounds, salts, polymorphs, hydrates, solvates and the like, is used herein, this is taken to mean also a single compound, salt, polymorph, isomer, hydrate, solvate or the like.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Types of 17α-Hydroxylase/C_{17,20}-Lyase (CYP17) Inhibitors

Certain 17α-hydroxylase/C_{17,20}-lyase (CYP17) inhibitors are described in U.S. Pat. No. 5,604,213, which is herein incorporated by reference in its entirety. In certain embodiments, the CYP17 inhibitor can be, without limitation, abiraterone or metabolites, analogs, derivatives or pharmaceutical acceptable salts thereof.

In some embodiments, the CYP17 inhibitor can comprise

17-(3-pyridyl)androsta-5,16-dien-3ß-ol;

17-(3-pyridyl)androsta-3,5,16-triene;

17-(3-pyridyl)androsta-4,16-dien-3-one;

17-(3-pyridyl)estra-1,3,5[10],16-tetraen-3-ol;

17-(3-pyridyl)-5α-androst-16-en-3α-ol;

17-(3-pyridyl)-5α-androst-16-en-3-one;

17-(3-pyridyl)-androsta-4,16-diene-3,11-dione;

17-(3-pyridyl)-androsta-3,5,16-trien-3-ol;

6α- and 6ß-fluoro-17-(3-pyridyl)androsta-4,16-dien-3-one;

17-(3-pyridyl)androsta-4,16-dien-3,6-dione;

3α-trifluoromethyl-17-(3-pyridyl)androst-16-en-3ß-ol

or their acid addition salts and 3-esters as well as metabolites, analogs, derivatives or a pharmaceutical acceptable salts thereof.

In certain embodiments, the CYP17 inhibitor can have the structure of formula (I):

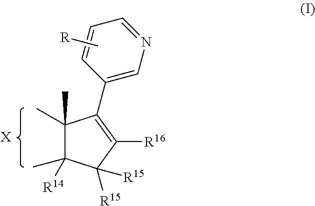

wherein X represents the residue of the A, B and C rings of a steroid which can be, without limitation, androstan-3α- or 3ß-ol; androst-5-en-3α- or 3ß-ol; androst-4-en-3-one; androst-2-ene; androst-4-ene; androst-5-ene; androsta-5,7-dien-3α or 3ß-ol; androsta-1,4-dien-3-one; androsta-3,5-diene; androsta-3,5-diene-3-ol; estra-1,3,5[10]-triene; estra-1,3,5 [10]-trien-3-ol; 5α-androstan-3-one; androst-4-ene-3,11-dione; 6-fluoroandrost-4-ene-3-one; or androstan-4-ene-3,6-dione; each of which, where structurally permissible, can be further derivatized in one or more of the following ways, including, but not limited to, to form 3-esters; to have one or more carbon or carbon ring double bonds in any of the 5,6-, 6,7-, 7,8-, 9,11- and 11,12-positions; as 3-oximes; as 3-methylenes; as 3-carboxylates; as 3-nitriles; as 3-nitros; as 3-desoxy derivatives; to have one or more hydroxy, halo, C₁₋₄-alkyl, trifluoromethyl, C₁₋₄-alkoxy, C₁₋₄-alkanoyloxy, benzoyloxy, oxo, methylene or alkenyl substituents in the A, B, or C-ring; or to be 19-nor;

R represents a hydrogen atom or an alkyl group of 1-4 carbon atoms;

R¹⁴ represents a hydrogen atom, a halogen atom or an alkyl group of 1 to 4 carbon atoms;

each of the R¹⁵ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group;

or R¹⁴ and one of the R¹⁵ groups together represent a double bond and the other R¹⁵ group represents a hydrogen atom or an alkyl group of 1 to 4 carbon atoms; and

R¹⁶ represents a hydrogen atom, halogen atom, or an alkyl group of 1 to 4 carbon atoms, in the form of the free bases or pharmaceutically acceptable acid addition salts.

Suitable inhibitors also include metabolites, derivatives, analogs, or pharmaceutically acceptable salts of formula (I).

CYP17 inhibitors suitable for the methods, compositions and combinations described here can be made according to any method known to one skilled in the art. For example, such inhibitors can be synthesized according to the method disclosed in U.S. Pat. Nos. 5,604,213 and 5,618,807 to Barrie et cil., herein incorporated by reference. Another method of making CYP17 inhibitors is disclosed in PCT Publication No. WO 2006/021777 to Bury, herein incorporated by reference.

In another embodiment, the CYP17 inhibitor can have the structure of formula (II):

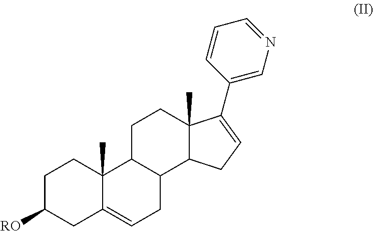

wherein R represents hydrogen or a lower acyl group having 1 to 4 carbon atoms. Suitable inhibitors also include metabolites, derivatives, analogs, or pharmaceutically acceptable salts of formula (II).

In still another embodiment, the CYP17 inhibitor can be a 3ß-alkanoyloxy-17-(3-pyridyl) androsta-5,16-diene in which the alkanoyloxy group has from 2 to 4 carbon atoms.

In a preferred embodiment, the CYP17 inhibitor comprises abiraterone, or metabolites, derivatives, analogs and pharmaceutically acceptable salts thereof. Without being limited by any theory, abiraterone is believed to act by inhibiting the production of testosterone precursors by blocking the conversion of pregnenolone to deydroepiandrosterone (DHEA) and progesterone to androstenedione.

In one embodiment, a pharmaceutically acceptable salt of abiraterone is abiraterone acetate, or 3ß-acetoxy-17-(3-pyridyl)androsta-5,16-diene, which is the 3-acetate and a pro-drug form of abiraterone, and it has the following structural formula (III):

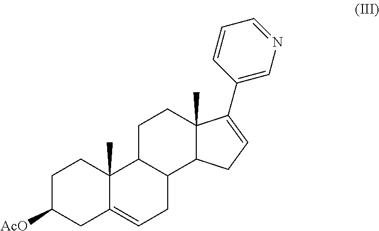 [SMILES: CC(=O)O[C@H]1CC[C@@]2(C)C(=CCC3C2CC[C@]2(C)C(c4cccnc4)=CCC32)C1]

wherein Ac refers to H₃C—C(═O)—Preferred salts of abiraterone, such as abiraterone acetate, and methods of making such salts, are also disclosed in U.S. Provisional Application No. 60/603,559 to Hunt and U.S. patent application Ser. No. 11/660,869 to Hunt, which are incorporated by reference in their entirety.

Preferred salts useful within the methods and compositions described herein include, but are not limited to, acetates, citrates, lactates, alkanesulfonates (e.g. methane-sulfonate or mesylate) and tartarates.

Of special interest is abiraterone acetate mesylate salt (i.e. 3ß-acetoxy-17-(3-pyridyl)androsta-5,16-diene mesylate salt) which has the following structural formula (IV):

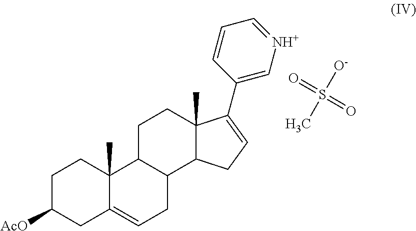

wherein Ac refers to H₃C—C(═O)—

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques already known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The invention also includes all suitable isotopic variations of a compound A of the invention. An isotopic variation of a compound A of the invention is defined as one in which at least one atom is replaced by an atom having the same atomic number but an atomic mass different from the atomic mass usually or predominantly found in nature. Examples of isotopes that can be incorporated into a compound A of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴, I²⁹¹ and ¹³¹I, respectively. Certain isotopic variations of a compound A of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound A of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds A of the present invention as single stereoisomers, or as any mixture of said stereoisomers, in any ratio. Isolation of a single stereoisomer, e.g. a single enantiomer or a single diastereomer, of a compound A of the present invention may be achieved by any suitable state of the art method, such as chromatography, especially chiral chromatography, for example.

The present invention includes all possible tautomers of the compounds A of the present invention as single tautomers, or as any mixture of said tautomers, in any ratio.

Furthermore, the present invention includes all possible crystalline forms, or polymorphs, of the compounds A of the present invention, either as single polymorphs, or as a mixture of more than one polymorphs, in any ratio.

Dosages of the 17α-Hydroxylase/C_{17,20}-Lyase (CYP17) Inhibitor

The therapeutically effective amounts or suitable dosages of the CYP17 inhibitor depend upon a number of factors, including the nature of the severity of the condition to be treated, the particular inhibitor, the route of administration and the age, weight, and response of the individual patient. Suitable daily dosages of CYP17 inhibitors can generally range, in single or divided or multiple doses, from about 10 mg/day to about 15000 mg/day, about 10 mg/day to about 10000 mg/day, about 10 mg/day to about 5000 mg/day, about 10 mg/day to about 2500 mg/day, about 10 mg/day to about 2000 mg/day, about 10 mg/day to about 1000 mg/day, from about 100 mg/day to about 15000 mg/day, from about 100 mg/day to about 10000 mg/day, trom about 100 mg/day to about 5000 mg/day, from about 100 mg/day to about 2500 mg/day, from about 100 mg/day to about 2000 mg/day, from about 100 mg/day to about 1000 mg/day, from about 250 mg/day to about 15000 mg/day, from about 250 mg/day to about 10000 mg/day, from about 250 mg/day to about 5000 mg/day, from about 250 mg/day to about 2500 mg/day, from about 250 mg/day to about 2000 mg/day, from about 250 mg/day to about 1000 mg/day, from about 800 mg/day to about 15000 mg/day, from about 800 mg/day to about 10000 mg/day, from about 800 mg/day to about 5000 mg/day, from about 800 mg/day to about 2500 mg/day, from about 800 mg/day to about 2000 mg/day, from about 1000 mg/day to about 15000 mg/day, from about 1000 mg/day to about 10000 mg/day, from about 1000 mg/day to about 5000 mg/day, from about 1000 mg/day to about 2500 mg/day, or from about 1000 mg/day to about 2000 mg/day.

In some embodiments, the specific dosage of a CYP17 inhibitor per day, in single or divided or multiple doses, by any route of administration (such as oral administration) includes without limitation about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1250 mg, about 1500 mg, about 1750 mg, about 2000 mg, about 2250 mg, about 2500 mg, about 2750 mg, about 3000 mg, about 3250 mg, about 3500 mg, about 3750 mg, about 4000 mg, about 4250 mg, about 4500 mg, about 4750 mg, about 5000 mg, about 5250 mg, about 5500 mg, about 5750 mg, about 6000 mg, about 6250 mg, about 6500 mg, about 6750 mg, about 7000 mg, about 7250 mg, about 7500 mg, about 7750 mg, about 8000 mg, about 8250 mg, about 8500 mg, about 8750 mg, about 9000 mg, about 9250 mg, about 9500 mg, about 9750 mg, about 10000 mg, about 10250 mg, about 10500 mg, about 10750 mg, about 11000 mg, about 11250 mg, about 11500 mg, about 11750 mg, about 12000 mg, about 12250 mg, about 12500 mg, about 12750 mg, about 13000 mg, about 13250 mg, about 13500 mg, about 13750 mg, about 14000 mg, about 14250 mg, about 14500 mg, about 14750 mg, about 15000 mg, about 15250 mg, about 15500 mg, about 15750 mg, about 16000 mg, about 17000 mg, about 18000 mg, about 19000 mg, or about 20000 mg.

Also, in some embodiments, the therapeutically effective amount of the CYP17 inhibitor may be administered once per day. In other embodiments, the CYP17 inhibitor is administered more than once per day. Also, the frequency in which any of these inhibitors can be administered can be once or more than once, (e.g. twice, 3 times, 4 times, etc.) per about 2 days, about 3 days, about 4 days, about 5 days, about 6 days, about 10 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every 12 months, about every year, about every 2 years, about every 3 years, about every 4 years, or about every 5 years.

Furthermore, the above frequencies of administration can occur continuously or non-continuously over certain time periods. For example, a certain amount of a CYP17 inhibitor can be administered daily continuously over 28 days.

Time periods over which the frequencies of administration can occur continuously or noncontinuously include without limitation about 1 day, about 2 days, about 3 days, about 4 days, about 5 days, about 6 days, about 10 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every 12 months, about every year, about every 2 years, about every 3 years, about every 4 years, or about every 5 years.

In some embodiments, the therapeutically effective amount of the CYP17 inhibitor is administered using dose cycling or a dosing regimen in which the CYP17 inhibitor is administered at a certain frequency, such as those discussed above, during a certain treatment period of a particular time duration, such as those described above. The treatment period is then followed by a nontreatment period of a certain time duration, such as the time periods described above, in which the CYP17 inhibitor is not administered to the patient. In certain embodiments, no CYP17 inhibitor is administered during the non-treatment period. In other embodiments, another CYP17 inhibitor is administered during the non-treatment period.

This non-treatment period can then be followed by aseries of subsequent treatment and non-treatment periods of the same or different frequencies or the same or different lengths of time. In some embodiments, the treatment and non-treatment periods are altemated. In other embodiments, a first treatment period in which a first amount of the CYP17 inhibitor is administered can be followed by another treatment period in which a same or different amount ofthe same or a different CYP17 inhibitor is administered. The second treatment period can be followed by other treatment period. During the treatment and non-treatment periods, one or more additional therapeutic agents can be administered to the patient.

Methods of Administration of the CYP17 Inhibitor

The CYP17 inhibitor can be administered by any method known to one skilled in the art. The CYP17 inhibitor can be administered in the form of a composition, in one embodiment a pharmaceutical composition, such as those described below. Preferably the composition containing the CYP17 inhibitor is pharmaceutically suitable for oral administration.

Examples of modes of administration include parenteral (e.g., subcutaneous, intramuscular, intraorbital, intracapsular, intraspinal, intrasternal, intravenous, intradermal, intraperitoneal, intraportal, intra-arterial, intrathecal, transmucosal, intra-articular, and intrapleural,), transdermal (e.g., topical), epidural, and mucosal (e.g., intranasal) injection or infusion, as well as oral, inhalation, pulmonary, and rectal administration.

The CYP17 inhibitor can be administered at various times during the course of the day, e.g., in the morning or in the evening. In some embodiments, the CYP17 inhibitor is administered with food. This means that the CYP17 inhibitor is taken by the patient while ingesting food, immediately after consumption of food by the patient, or immediately before consumption of food by the patient. In other embodiments, the inhibitor is administered about 1 minute to about 1 hour after consumption of food by the patient. In other embodiments, the CYP17 inhibitor is administered about 1 minute to about 30 minutes after consumption of food by the patient. The CYP17 lyase inhibitor can be administered with food at the frequencies and over the same time periods as discussed above. Also, the CYP17 inhibitor can be administered with food in a dosing regimen such as those described above. For example, in the one embodiment, the CYP17 inhibitor is administered once per day with food continuously during a first treatment cycle of about 28 days.

Alternatively, the CYP17 inhibitor can also be administered during periods of fasting. In some embodiments, the CYP17 inhibitor is administered first thing in the morning, before any food has been consumed by the patient. In certain embodiments, the inhibitor is administered after the patient has fasted for less than about 5 hours. In other embodiments, the CYP17 inhibitor is administered after the patient has fasted for less than about 2 hours. The CYP17 inhibitor can be administered after fasting at the frequencies and over the same time periods as discussed above. Furthermore, the CYP17 inhibitor can be administered after fasting and during a dosing regimen such as those described above.

Compositions Containing a CYP17 Inhibitor

In certain embodiments, the compositions according to the present invention contain a CYP17 inhibitor, preferably abiraterone acetate. The compositions can take various forms such as, but not limited to, solutions, suspensions, emulsions, tab lets, pills, capsules, powders or sustained-release formulations, depending on the intended route of administration.

For topical or transdermal administration, the compositions can be formulated as solutions, gels, ointments, creams, suspensions or salves.

For oral administration, the compositions may be formulated as tablets, pills, dragees, troches, capsules, liquids, gels, syrups, slurries, suspensions and the like, for oral ingestion by a patient to be treated.

The composition may also be formulated in rectal or vaginal compositions such as suppositories or retention enemas that contain conventional suppository bases such as cocoa butter or other glycerides.

In addition to the formulations described previously, the composition may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

Additionally, the composition may be delivered using a sustained-release system, such as semi-permeable matrices of solid polymers containing the compositions.

Various forms of sustained-release materials have been established and are well known by those skilled in the art. Sustained-release capsules, depending on their chemical nature, can release the composition over a period of hours, days, weeks, or months. For example, a sustained release capsule can release the compositions over a period of 100 days or longer.

Depending on the chemical nature and the biological stability of the composition, additional strategies for stabilization may be employed.

The compositions can further comprise a pharmaceutically acceptable carrier. The term “carrier” refers to a diluent, adjuvant (e.g., Freund's adjuvant (complete and incomplete)), excipient, or vehicle with which the therapeutic is administered.

For parenteral administrations, the composition can comprise one or more of the following carriers: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

For oral solid formulations suitable carriers include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, fats and oils; granulating agents; and binding agents such as microcrystalline cellulose, gum tragacanth or gelatin; disintegrating agents, such as cross-linked polyvinylpyrrolidone, agar, or alginic acid or a salt thereof such as sodium alginate, Primogel, or corn starch; lubricants, such as magnesium stearate or Sterotes; glidants, such as colloidal silicon dioxide; a sweetening agent, such as sucrose or saccharin; or flavoring agents, such as peppermint, methyl salicylate, or orange flavoring. If desired, solid dosage forms may be sugar-coated or enteric-coated using standard techniques.

Pharmaceutically Acceptable Salts of the Alkaline-Earth Radionuclide Radium-223

A suitable pharmaceutically acceptable salt of radium-223 may be, for example, an acid addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

The present invention includes the use of the nuclide radium-233 as a cationic species and/or associated to a chelator or another form of a carrier molecule with affinity for calcified tissues. This also includes, but is not limited to the combination of radium-223 with a chelator that can be subsequently conjugated to a molecule with affinity for calcified tissues. The intent is to use the radioisotope to generate a cascade of α-particles on bone surfaces and/or in calcified tumors for the palliation of pain caused by various diseases and/or for the prophylactic use against possible minimal disease to the skeleton, and/or also for the therapeutic treatment of established cancer to the bone.

A preferred suitable pharmaceutically acceptable salt of radium-223 is the dichloride (Ra²²³Cl₂).

Methods for preparation of a physiologically acceptable solution comprising radium-223 are disclosed e.g. in WO 2000/40275(A2), WO 2011/134671(A1), and WO 2011(134672(A1).

Compositions Containing Radium-223

Physiologically acceptable solutions comprising radium-223 show a unique mechanism of action as a targeted radiopharmaceutical. They represent a new generation of alpha emitting therapeutic pharmaceuticals based on the natural bone-seeking nuclide radium-223.

The physiologically acceptable preparation for in vivo administration according to the present invention comprises dissolved radium-223 salt, with or without a single or a combination of several cations, as stabilizing alkaline earth metal cation analogue carrier, with or without an agent to prevent precipitation and/or generation of colloids, in addition to pharmacologically acceptable carriers and adjuvans.

The cation acting as stabilizing alkaline earth metal cation can be selected from the group consisting of magnesium, calcium and strontium. Furthermore, the agent to prevent precipitation and/or generation of colloids is a carboxylic acid or a combination of carboxylic acids, such as oxalic acid, oxaloacetic acid, tartaric acid, succinic acid, malic acid and malonic acid.

Preferably, an aqueous solution of radium-223 chloride (²²³RaCl2) for intravenous injection, sterile and free from bacterial endotoxins is used.

Preferably, the solution is isotonic, containing a sodium citrate buffered saline to physiological pH.

Methods of Administration of Radium-223

The ²²³Ra salt or derivative thereof will be administered to a mammal, such as a human, in need thereof by all available administration routes, such as oral, subcutaneous, intravenous, intraarterial or transcutane. Preferably the active compound is administered by injection or infusion.

Oral administration is performed by use of tablets, capsules, powders or in liquid form, such as suspension, solution, syrup or emulsion. When formed into tablets conventional expicients, lubricating agents and binding agents are used.

When administered as liquids conventional liquid carriers are used.

When administered as injection or infusion solutions the carrier is preferably isotonic saline, with or without agent(s) to stabilize the radium cation to prevent precipitation of radium salts or insoluble complexes.

Preferably, radium-223 is administered intravenously by qualified personnel as a slow bolus injection. An intravenous access line should be used for administration of radium-223. The line should be flushed with isotonic saline before and after injection of radium-223.

Dosages of Radium-223

The concentrations of the compounds in the preparation will generally be less than the individual LD₅₀ dose, for example less than 20% of the LD₅₀ dose, and thus vary for the different components.

The activity of ²²³Ra will be dependent upon the type and route of administration and the underlying condition or disease and will vary between approximately 50 kBq to approximately 10 MBq, administered in single or multiple doses for mammals, such as for example humans.

A preferred dosage regimen for radium-223 chloride injection is 50 kBq per kg body weight given at 4 week intervals, as a course consisting of 6 injections. Single radium-223 doses up to 250 kBq per kg body weight were evaluated in a phase I clinical trial. The observed adverse reactions at this dose were diarrhea and reversible myelosuppression (including one case (1/5) of grade 3 neutropenia).

As an example, the aqueous radium-223 dichloride solution may be supplied in a single-dose 10 ml vial which contains a fill volume of 6 ml. This product has a radioactivity concentration of radium-223 of 1,000 kBq/mL (0.03 mCi/mL), corresponding to 0.53 ng/mL of radium at reference date. The active moiety is the alpha particle emitting nuclide radium 223 (half-life is 11.4 days), present as a divalent cation (223Ra2+). The fraction of energy emitted from radium-223 and its daughters as alpha-particles is 95.3%, the fraction emitted as beta-particles is 3.6%, and the fraction emitted as gamma-radiation is 1.1%. The combined energy from the emitted radiation from complete decay of radium-223 and its daughter nuclides is 28.2 MeV.

Radium-223 selectively targets areas of increased bone turnover, as in bone metastases, and concentrates by forming a complex with hydroxyapatite. Alpha emission contributes about 93% of the total radiation absorbed dose. The high linear energy alpha particle radiation induces double-strand DNA breaks, resulting in a potent and localized cytotoxic effect in the target areas containing metastatic cancer cells. The short path length (less than 100 micrometers) of the alpha particles minimizes the effect on adjacent healthy tissue such as the bone marrow.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Combinations and Kits According to the Present Invention

In accordance with an embodiment, the present invention relates to a combination of any compound A mentioned herein with any compound B mentioned herein.

Further, the present invention relates to:

a kit comprising:
a combination of:

component A: one or more CYP17 inhibitors, or a physiologically acceptable salt, solvate, hydrate or stereoisomer thereof;

component B: a suitable pharmaceutically acceptable salt of the alkaline-earth radionuclide radium-223 or a solvate or a hydrate thereof; and, optionally,

component C: one or more further pharmaceutical agents;

in which optionally either or both of said components A and B in any of the above-mentioned combinations are in the form of a pharmaceutical formulation which is ready for use to be administered to a patient.

The combinations and the kits of the present invention may be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

Preferred use of the combination and kit is the treatment of breast and prostate cancer, especially CRPC and bone metastases.

Combinations and kits of the present invention might be utilized to inhibit, block, reduce, decrease, etc., cell proliferation and/or cell division, and/or produce apoptosis.

This invention includes a method comprising administering to a mammal in need thereof, including a human, an amount of a compound A and an amount of compound B of this invention, or a pharmaceutically acceptable salt, isomer, polymorph, metabolite, hydrate, solvate or ester thereof; etc. which is effective to treat the disorder.

Hyper-proliferative disorders include but are not limited, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

Combinations of the present invention might also be used for treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer. Thus, combinations of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

The combinations and kits of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

Compounds A and B can be administered as the sole pharmaceutical agents or in combination with one or more further pharmaceutical agents C where the resulting combination of A, B and C causes no unacceptable adverse effects. For example, the combinations of A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

Component C, can be one or more pharmaceutical agents such as 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, paliferm in, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib , regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tam ibarotene, tamoxifen, tasonerm in, teceleukin, tegafur, tegafur+gimeracil+oteracil, temoporfin, temozolomide, temsirolimus, teniposide, testosterone, tetrofosm in, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin or combinations thereof.

Alternatively, said component C can be one or more further pharmaceutical agents selected from gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti-hyper-proliferative agents which can be added as component C to the combination of A and B of the present invention include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use as component C with the combination of compounds A and B of the present invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel (when component B is not itself paclitaxel), pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use as component C with the combination of compounds A and B of the present invention include but are not limited to other anti-cancer agents such as epothilone and its derivatives, irinotecan, raloxifen and topotecan.

Generally, the use of cytotoxic and/or cytostatic agents as component C in combination with a combination of compounds A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(8) provide a longer time for tumor progression, and/or
(9) yield efficacy and tolerability results at least as good as those of the agents used alone, compared to known instances where other cancer agent combinations produce antagonistic effects.

1. A pharmaceutical combination comprising compounds A and B, wherein compound A is a CYP17 inhibitor, and compound B is a pharmaceutically acceptable salt of the alkaline-earth radionuclide radium-223.

2. The combination according to claim 1, wherein the pharmaceutically acceptable salt of the alkaline-earth radionuclide radium-223 is radium-223 dichloride.

3. The combination according to claim 1, wherein the compound A is abiraterone acetate.

4. A method for the treatment of disorder selected from breast cancer, prostate cancer, hepatocyte carcinoma, lung cancer, non-small cell lung carcinoma, colorectal cancer, melanoma, pancreatic cancer and metastases thereof comprising administering to a patient in need thereof a therapeutically effective amount of the combination according to claim 1.

5. The method according to claim 4 wherein the disorder is selected from breast cancer, prostate cancer and metastases thereof.

6. The method according to claim 4 wherein the metastases are bone metastases.

7. A method of for the treatment of hepatocyte carcinoma, lung cancer, non-small cell lung carcinoma, colorectal cancer, melanoma, pancreatic cancer or breast cancer, in a subject in need thereof, comprising administering to said subject a therapeutically effective amount of the combination according to claim 1.

8. A kit comprising the combination of: according to claim 1;
and, optionally, one or more further pharmaceutical agents C;
wherein optionally both or either of said components A and B are in the form of a pharmaceutical formulation which is ready for use to be administered simultaneously, concurrently, separately or sequentially.

9. The pharmaceutical composition containing a combination according to claim 1 together with one or more pharmaceutically acceptable excipients.

10. The pharmaceutical combination of claim 1 wherein the pharmaceutically acceptable salt of the alkaline-earth radionuclide radium-223 is associated with a chelator.
